(19) United States
(12) Patent Application Publication
(10) Pub. No.: US 2022/0354725 A1
(43) Pub. Date: Nov. 10, 2022

(54) SPINAL ORTHOPEDIC EXERCISE EQUIPMENT

(71) Applicant: Woo-Yeol SHIN, Seoul (KR)

(72) Inventors: Woo-Yeol SHIN, Seoul (KR); Hyun-Ki KWON, Seoul (KR)

(21) Appl. No.: 17/619,948

(22) PCT Filed: Oct. 12, 2020
(86) PCT No.: PCT/KR2020/013874

§ 371 Date: Dec. 16, 2021

(30) Foreign Application Priority Data
Nov. 26, 2019 (KR) 10-2019-0153001

(51) Int. Cl. A61H 1/02; A61H 7/00

(52) U.S. Cl. CPC A61H 1/0218; A61H 7/001; A61H 2201/1623; A61H 2201/1695; A61H 2201/1261; A61H 2201/1676

(57) ABSTRACT

Proposed is spinal orthopedic exercise equipment which can obtain an improved exercise effect related to spinal orthopedics with a simple structure. The spinal orthopedic exercise equipment supporting a user's back and waist to apply pressure thereto includes: a first body having a plurality of first protrusions protruding therefrom, a second body coupled to the first body and having a plurality of second protrusions protruding therefrom, a flexible connector located between the first body and the second body to be bendably coupled thereto, and a removable rolling bar coupled to a bottom surface of the first body or the second body having no protrusion and supporting the first body or the second body, the removable rolling bar causing the first body or the second body to perform a seesaw movement relative to a support point thereof.

### TECHNICAL FIELD

The present disclosure relates generally to spinal orthopedic exercise equipment. More particularly, the present disclosure relates to spinal orthopedic exercise equipment which can obtain an improved exercise effect related to spinal orthopedics with a simple structure.

### BACKGROUND ART

Modern people suffer from various pains occurring in various parts of the body. Various causal actions such as the excessive use of joints and the maintaining of incorrect postures are routinely repeated, thus causing chronic pain. This pain is often not easily relieved.

Particularly, imbalance of the spine supporting the entire body or pain caused by the imbalance has a great influence on an individual to the extent that the individual's daily life is difficult. Various issues of muscle pain, neuralgia, scoliosis, and a herniated disk which occur at the neck supporting the head, the back, and the side of the spine leading to the waist are not easily treated even by surgery, and cause extreme pain, so more active measures are required.

One of known countermeasures is to improve the abnormality of the spine through exercise, and to this end, various exercise methods, assistive devices, and correctors have been developed. For example, exercise equipment disclosed in Korean Patent No. 10-1142888 is used to perform such a countermeasure.

However, conventionally known exercise equipment or correctors have complicated structures and are difficult to be installed. Furthermore, the exercise equipment or correctors occupy much space, and thus are difficult to be used on a daily basis in a user's home. In addition, the actual exercise effect of the exercise equipment or correctors is less than expected, and thus such equipment or correctors have many shortcomings in terms of effectiveness. Therefore, this is required to be improved.

### DOCUMENT OF RELATED ART

### Patent Document

(Patent Document 1) Korean Patent No. 10-1142888, (2012. 05. 09), Specification

### DISCLOSURE

### Technical Problem

To solve the above problems, the present disclosure is intended to propose spinal orthopedic exercise equipment which can obtain an improved exercise effect related to spinal orthopedics with a simple structure.

The technical objective of the present disclosure is not limited to the above-mentioned objective, and other objectives not mentioned will be clearly understood by those skilled in the art from the following description.

### Technical Solution

Spinal orthopedic exercise equipment supporting a user's back and waist to apply pressure thereto includes: a first body having a plurality of first protrusions protruding therefrom, a surface of the first body on which the first protrusions are disposed being configured as a curved surface, a second body coupled to the first body and having a plurality of second protrusions protruding therefrom, a surface of the second body on which the second protrusions are disposed being configured as a curved surface different from the curved surface of the first body, a flexible connector located between the first body and the second body to be bendably coupled thereto, and a removable rolling bar coupled to a bottom surface of the first body or the second body having no protrusion and supporting the first body or the second body, the removable rolling bar causing the first body or the second body to perform a seesaw movement relative to a support point thereof.

In the spinal orthopedic exercise equipment, a position of each of the first body and the second body may change according to the bending of the flexible connector.

The first body may include a convex surface, and the second body may include a concave surface, wherein bending directions of the curved surfaces of the first body and the second body may be reversed to each other along a longitudinal direction in which the first body and the second body are connected to each other.

The curved surface of the first body may face and support a user's waist, and the curved surface of the second body may face and support the user's back.

The flexible connector may be configured as an elastic body which connects the curved surface of the first body to the curved surface of the second body by a continuous surface thereto.

In the spinal orthopedic exercise equipment, the flexible connector, a first curved part in which the curved surface of the first body is formed, and a second curved part in which the curved surface of the second body is formed may be configured to be integrated with each other.

Each of the first body and the second body may include a curved part having a curved surface formed thereon and having protrusions protruding therefrom, and a bottom part coupled to the curved part and constituting a bottom surface having no protrusion, wherein the curved part and the bottom part may include a sliding coupling part configured as a pair of coupling protrusion and fitting groove which are slidably coupled to each other.

The curved part and the bottom part may further include a fastening point combination part which forms a fastening point at one point by coupling parts facing each other to each other which are located at other sides at which the pair of coupling protrusion and fitting groove is not formed.

The removable rolling bar may have an outer circumferential surface configured as a convex curved surface, wherein the outer circumferential surface may not be in contact with the bottom surface of the first body or the second body.

The removable rolling bar may include a cylindrical coupling protrusion protruding perpendicularly to the removable rolling bar on a surface thereof in contact with the bottom surface of the first body or the second body.

The spinal orthopedic exercise equipment may further include: a cylindrical coupling hole formed in the bottom surface of each of the first body and the second body and receiving the cylindrical coupling protrusion, and stoppers formed respectively on the cylindrical coupling hole and an outer side of the cylindrical coupling protrusion, the stoppers being engaged with each other to restrict a rotation of the removable rolling bar and to fasten the removable rolling bar perpendicularly to a longitudinal direction in which the first body and the second body are connected to each other.

The first body and the second body may include respectively hollow parts which are spaces in which protrusions are not formed, the hollow parts being located respectively between the first protrusions and between the second protrusions and respectively along centers of the first body and the second body in a longitudinal direction in which the first body and the second body are connected to each other.

The spinal orthopedic exercise equipment may further include: a plurality of connection plates arranged by intersecting with a longitudinal direction in which the first body and the second body are connected to each other, each of the connection plates formed between the first protrusions and between the second protrusions so as to connect at least two protrusions of the first protrusions to each other and to connect at least two protrusions of the second protrusions to each other.

### Advantageous Effects

The spinal orthopedic exercise equipment of the present disclosure is used to perform exercises related to spinal orthopedics very conveniently regardless of location. The exercise equipment of the present disclosure has a simple structure and is easy to be carried, so the exercise equipment not only has advantages in use and storage, but also maximizes a user's exercise effect through a structure which induces the contraction and elongation of the spine and surrounding portions thereof. In addition, the exercise equipment may be used by changing a part of a structure thereof or freely changing an arrangement thereof as needed, thereby embodying various exercise methods and providing improved exercise effects according thereto. Furthermore, the exercise equipment is in close contact with a user's body curves and transforms according to the body's movement, which can induce the body's movement, thereby enabling even a beginner to easily and effectively exercise.

### DESCRIPTION OF DRAWINGS

FIG. 1 is a perspective view of spinal orthopedic exercise equipment according to an embodiment of the present disclosure.

FIG. 2 is a perspective view of the exercise equipment of FIG. 1 viewed in a different direction.

FIG. 3 is a view illustrating the process of attaching/detaching a removable rolling bar of the exercise equipment of FIG. 1.

FIG. 4 is an exploded view of the exercise equipment of FIG. 1.

FIG. 5 is a top plan view of the exercise equipment of FIG. 1.

FIG. 6 is a view illustrating the change of the shape of the exercise equipment according to the bending of a flexible connector by the side view of the exercise equipment of FIG. 1.

FIG. 7 is a front view of the exercise equipment of FIG. 1.

FIG. 8 is enlarged perspective views of the flexible connector of the exercise equipment of FIG. 1 when the flexible connector is viewed in different directions.

FIG. 9 is a view illustrating the method of using of the exercise equipment of FIG. 1.

FIGS. 10 to 13 are views illustrating the operation process of the exercise equipment of FIG. 1.

### MODE FOR INVENTION

Advantages and features of the present disclosure and methods for achieving them will become apparent with reference to an embodiment described later in detail in conjunction with the accompanying drawings. However, the spinal orthopedic exercise equipment of the present disclosure is not limited to the embodiment disclosed below, but may be embodied in various forms. Only the present embodiment is provided so that the present disclosure is complete and to completely inform the scope of the present disclosure to those skilled in the art to which the present disclosure belongs, and the present disclosure is only defined by the claims. Like reference numerals refer to like elements throughout this specification.

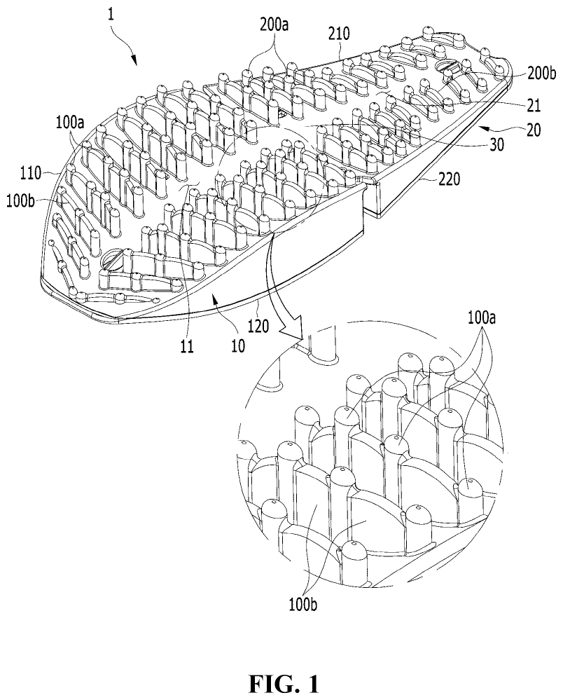

Hereinafter, the spinal orthopedic exercise equipment according to the embodiment of the present disclosure will be described in detail with reference to FIGS. 1 to 13.

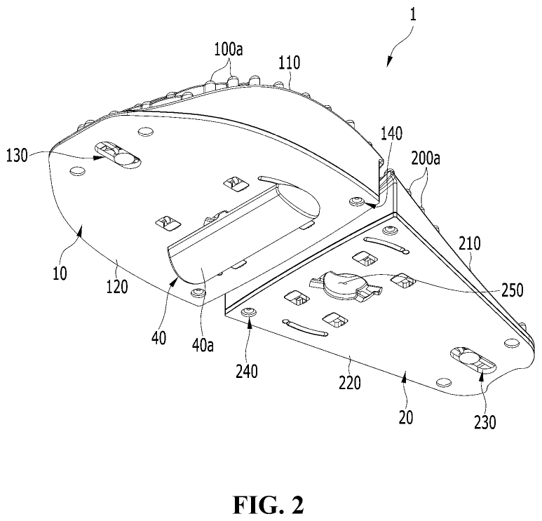

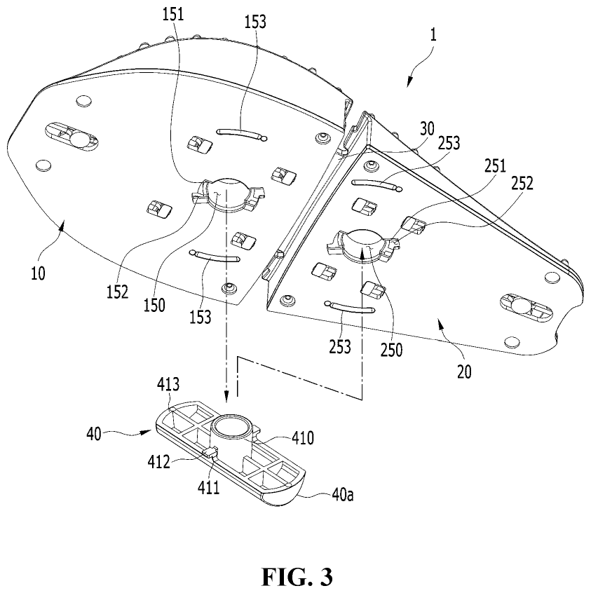

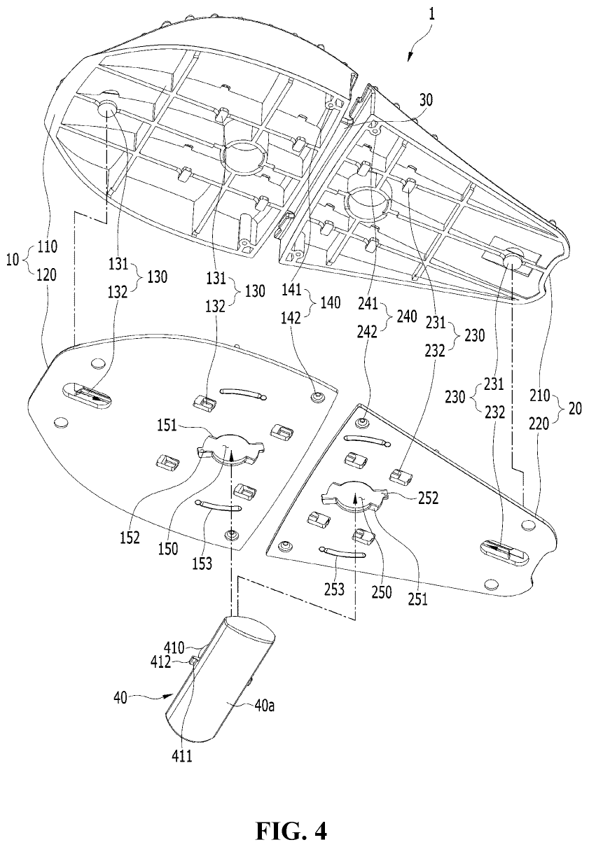

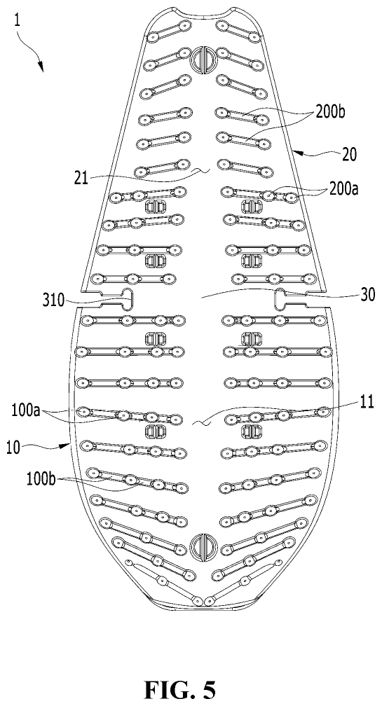

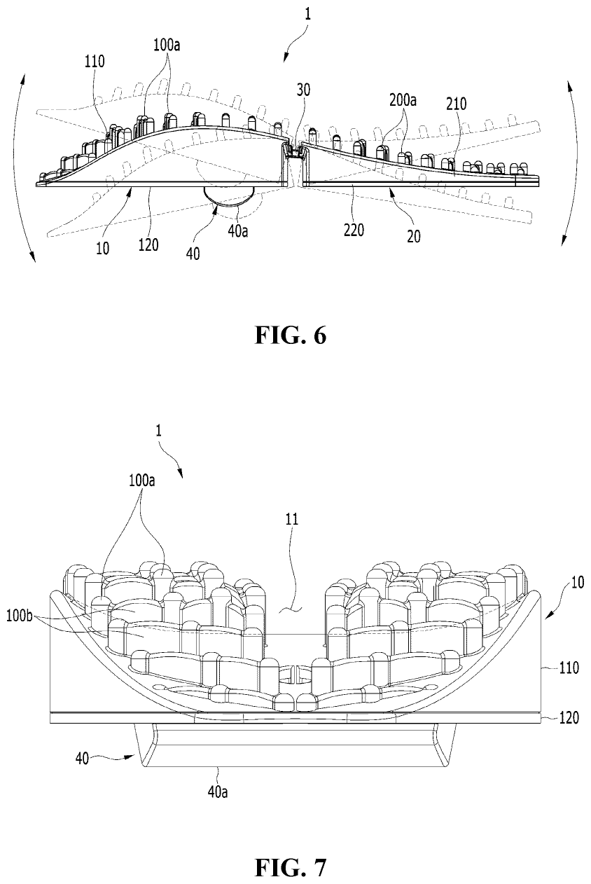

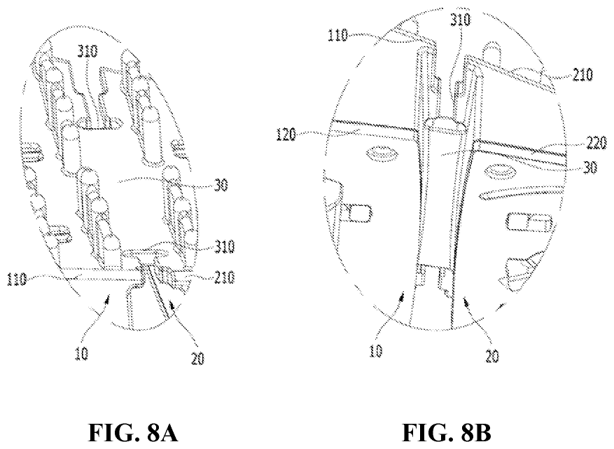

FIG. 1 is a perspective view of the spinal orthopedic exercise equipment according to the embodiment of the present disclosure; FIG. 2 is a perspective view of the exercise equipment of FIG. 1 viewed in a different direction; FIG. 3 is a view illustrating the process of attaching/detaching a removable rolling bar of the exercise equipment of FIG. 1; FIG. 4 is an exploded view of the exercise equipment of FIG. 1; FIG. 5 is a top plan view of the exercise equipment of FIG. 1; FIG. 6 is a view illustrating the change of the shape of the exercise equipment according to the bending of a flexible connector by the side view of the exercise equipment of FIG. 1; and FIG. 7 is a front view of the exercise equipment of FIG. 1.

Referring to FIGS. 1 to 7, the spinal orthopedic exercise equipment 1 of the present disclosure has a structure in which different bodies [a first body 10 and a second body 20] having protrusions formed on the outer surface thereof are connected to each other by the flexible connector 30, and the first body 10 and the second body 20 move relatively to each other relative to the flexible connector 30 to change positions thereof (see FIG. 6). Due to such a structure, when the spinal orthopedic exercise equipment 1 of the present disclosure is in close contact with a body, the spinal orthopedic exercise equipment 1 may be flexibly transformed according to the movement of the body and may continuously maintain close contact with different points of the body such as the waist and back. Furthermore, the spinal orthopedic exercise equipment 1 may be transformed to apply stimulation to the spine of the body in various manners.

In addition, in the spinal orthopedic exercise equipment 1 of the present disclosure, any one of the first body 10 and the second body 20 is coupled to the removable rolling bar 40 such that the first body 10 and the second body 20 are placed asymmetrically to each other, which naturally induces a user's posture change. Accordingly, in particular, the first body 10 and the second body 20 can change a user's posture to maximize an exercise effect in a very dynamic way accompanied by a seesaw movement relative to the removable rolling bar 40, rather than in the static way of staying still in one position. Such an effect is caused by a structure in which different parts of the first body 10 and the second body 20 are connected by the flexible connector 30 so as to be movable, and further, by a structure which includes the removable rolling bar 40 coupled asymmetrically to a side of the first body 10 or the second body 20 and inducing an unstable state. The effect can be considered to be a synergistic effect due to the complex action of these structures.

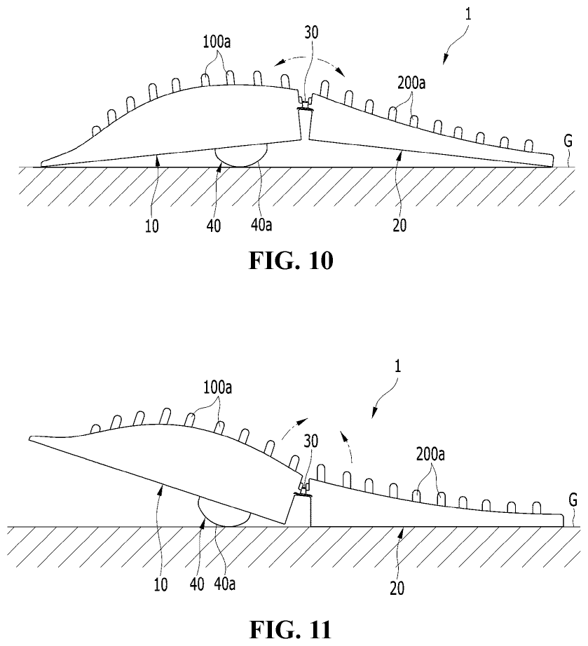

The spinal orthopedic exercise equipment 1 of the present disclosure is specifically configured as follows. The spinal orthopedic exercise equipment 1 is a spinal orthopedic exercise equipment which supports a user's back and waist to apply pressure thereto, and includes: the first body 10 having a plurality of first protrusions 100a protruding therefrom, a surface of the first body on which the first protrusions 100a are disposed being configured as a curved surface; the second body 20 coupled to the first body 10 and having a plurality of second protrusions 200a protruding therefrom, a surface of the second body on which the second protrusions 200a are disposed being configured as a curved surface different from the curved surface of the first body 10; the flexible connector 30 located between the first body 10 and the second body 20 to be bendably connected thereto; and the removable rolling bar 40 coupled to the bottom surface of the first body 10 or the second body 20 having no protrusion and supporting the first body 10 or the second body 20, the removable rolling bar causing the first body 10 or the second body 20 to perform a seesaw movement relative to a support point thereof. In the embodiment, the first body 10 includes a convex surface, and the second body 20 includes a concave surface, and the bending directions of the curved surfaces of the first body 10 and the second body 20 may be reversed to each other along a longitudinal direction in which the first body 10 and the second body 20 are connected to each other (see FIG. 6), and the curved surface of the first body 10 may face and be supported by a user's waist, and the curved surface of the second body 20 may face and be supported by the user's back (see FIG. 9). Hereinafter, the various characteristics of the spinal orthopedic exercise equipment 1 of the present disclosure such as the structure and function thereof will be described in more detail on the basis of the embodiment of the present disclosure with reference to each drawing.

First, the spinal orthopedic exercise equipment 1 of the present disclosure can be used to apply stimulation to the side of a spine and can be used by being in close contact with body parts at the periphery of the spine. Particularly, the spinal orthopedic exercise equipment 1 can support a user's back and waist to apply pressure thereto. In this case, the spinal orthopedic exercise equipment 1 is not limited to the back and waist. The spinal orthopedic exercise equipment 1 of the present disclosure may be disposed and used at various positions such as at least a portion of each of the back and waist and/or peripheral portions thereof. For example, the spinal orthopedic exercise equipment 1 of the present disclosure may be appropriately disposed in at least a portion of each of a part at which the thoracic vertebra is located and a part at which the lumbar vertebra is located or in a part corresponding to the peripheral portion thereof and may be used to apply stimulation to the side of the spine.

The first body 10 has the plurality of first protrusions 100a formed on an outer surface thereof by protruding therefrom. As illustrated in FIGS. 1 to 7, a surface on which the first protrusions 100a are disposed may be configured as a curved surface, and in this way, the surface on which the curved surface is formed may be a part which faces a user's body. In the embodiment, the first body 10 includes a convex surface and the second body 20 includes a concave surface, and the convex and concave surfaces are connected to each other and may form a curved surface which three-dimensionally changes in the longitudinal direction in which the first body 10 and the second body 20 are connected to each other. As illustrated in FIG. 6, the bending directions of the curved surfaces of the first body 10 and the second body 20 may be reversed to each other along a longitudinal direction in which the first body 10 and the second body 20 are connected to each other. In this case, the longitudinal direction in which the first body 10 and the second body 20 are connected to each other may correspond to the longitudinal direction of the spine. Accordingly, the first body 10 and the second body 20 may be configured in a three-dimensional curved shape corresponding to the curvature of the spine and may support the spine to apply stimulation thereto.

The second body 20 also has the plurality of second protrusions 200a protruding on the outer surface thereof. The surface on which the second protrusions 200a is disposed is also configured as a curved surface, and the surface on which the curved surface is formed may be a part which faces a user's body. As described above, the second body 20 may include a concave surface, and the concave surface may be connected to the convex surface of the first body 10 (see FIG. 6). That is, the first body 10 and the second body 20 are connected to each other by the flexible connector 30 to be described later and have different curved surfaces, respectively, formed thereon to form a three-dimensional shape. By using such a three-dimensional shape, the first body 10 and the second body 20 can be in closer contact with a user's body to apply stimulation thereto. For example, the curved surface of the first body 10 in which a convex surface is included faces a user's waist side, and the curved surface of the second body 20 in which a concave surface is included faces the user's back side so as to support the user's body more closely.

The first protrusions 100a of the first body 10 and the second protrusions 200a of the second body 20 have multiple protrusions disposed respectively on the curved surfaces of the first and second bodies. These protrusions may protrude and thus may be disposed in directions toward a body when the spinal orthopedic exercise equipment 1 supports the body. In the embodiment, the first protrusions 100a and the second protrusions 200a protrude parallel to each other in the same directions, but are not limited thereto, and when required, at least some of the protrusions may be arranged to have directions different from the directions of the other protrusions. The first protrusions 100a and the second protrusions 200a may be formed to have the same shapes as a whole, but may have different lengths according to positions thereof. For example, each of the first protrusions 100a and the second protrusions 200a may have a length decreasing gradually toward the end portion of each of the first body 10 and the second body 20 in the longitudinal direction. Additionally, each of the first body 10 and the second body 20 may include a concave surface in a width direction [in a direction perpendicular to the longitudinal direction in which the first body 10 and the second body 20 are connected to each other, that is, in the horizontal direction of FIG. 7], and because of this, a protrusion located at a center portion in the width direction may be formed to be relatively long, and a protrusion located at an outer side in the width direction may be formed to be relatively short (see FIG. 7). Particularly, the first body 10 includes a convex surface in the longitudinal direction described above and includes a concave surface in the width direction described above and thus may form a three-dimensional structure as a whole such as a horse saddle surface in which the convex and concave surfaces are combined with each other.

At least some of the first protrusions 100a may be connected to each other by connection plates 100b and at least some of the second protrusions 200a may be connected to each other by connection plates 200b. As illustrated in FIGS. 1 and 5, a connection plate 100b and a connection plate 200b are formed respectively between the first protrusions 100a and between the second protrusions 200a such that at least two first protrusions 100a are connected to each other by the connection plate 100b and at least two second protrusions 200a are connected to each other by the connection plate 200b. Accordingly, the connection plate 100b connects the first protrusions 100a to each other and the connection plate 200b connects the second protrusions 200a to each other. Furthermore, the connection plate 100b and the connection plate 200b may be arranged by intersecting with the longitudinal direction (a vertical direction of FIG. 5) in which the first body 10 and the second body 20 are connected to each other. The plurality of first protrusions 100a is connected to each other by the connection plates 100b and the plurality of second protrusions 200a is connected to each other by the connection plates 200b such that support forces of the first protrusions 100a and the second protrusions 200a are strengthened. Particularly, the deformations of the first protrusions 100a and the second protrusions 200a due to an external force may be minimized respectively in directions in which the connection plates 100b and 200b are arranged. Accordingly, even if large weight is applied to the first protrusions 100a and the second protrusions 200a, each of the first protrusions 100a and the second protrusions 200a can more securely maintain a shape thereof to support and stimulate a body.

The arrangement direction of the connection plate 100b or 200b may be different according to a position of the connection plate. For example, as illustrated in FIG. 5, there may be at least some of the connection plates 100b or 200b arranged slantingly along the longitudinal direction in which the first body 10 and the second body 20 are connected to each other. The connection plates 100b or 200b may be arranged more slantingly gradually toward the end portion of the first body 10 or the second body 20 in the longitudinal direction. The first protrusions 100a connected to each other by the connection plates 100b and the second protrusions 200a connected to each other by the connection plates 200b may respectively form small groups and may be arranged rectilinearly respectively in directions in which the connection plates 100b and 200b are arranged. As illustrated in FIG. 5, when the direction of each of the connection plates 100b or 200b changes, an arrangement direction thereof may change, and as the width of each of the connection plates 100b or 200b changes, the number of protrusions connected to each other by the connection plate may increase/decrease. Accordingly, due to the combined structure of the protrusions with the connection plates 100b and 200b, the support forces of the protrusions may be reinforced and the protrusions may be arranged in various patterns.

Accordingly, space in which protrusions are not arranged may be defined in a portion of each of the first body 10 and the second body 20 including protrusions. As illustrated in FIGS. 1 and 5, hollow parts 11 and 21 which are spaces in which protrusions are not formed may be formed respectively in the first body 10 and the second body 20, the hollow parts 11 and 21 being formed respectively between the first protrusions 100a and between the second protrusions 200a and respectively along the centers of the first body 10 and the second body 20 in the longitudinal direction in which the first body 10 and the second body 20 are connected to each other. When the spinal orthopedic exercise equipment 1 supports a body, the hollow parts 11 and 21 may overlap with a user's spine and may receive a part at which the spine is located. Accordingly, the hollow parts 11 and 21 prevent the spine from being pressed directly by the protrusions and protect the spine. The width or entire length of each of the hollow parts 11 and 21 may be appropriately changed as required.

Each of the first body 10 and the second body 20 may be configured as a structure in which a curved part having protrusions formed thereon and a bottom part are coupled to each other. That is, as illustrated in FIGS. 1 to 4, each of the first body 10 and the second body 20 may include the curved part [a first curved part 110 and a second curved part 210] and the bottom part [a first bottom part 120 and a second bottom part 220] coupled to the curved part and constituting a bottom surface having no protrusion. The first body 10 may be formed by the coupling of the first curved part 110 to the first bottom part 120, and the second body 20 may be formed by the coupling of the second curved part 210 to the second bottom part 220. Each of the first bottom part 120 and the second bottom part 220 is formed in a flat shape and may be supported on a flat part, such as a floor, when in use. The flexible connector 30 may be located between the first curved part 110 and the second curved part 210 to be connected thereto. Except for the parts of the first curved part 110 and the second curved part 210 connected to each other by the flexible connector 30, the first body 10 and the second body 20 may be spaced apart from each other.

Accordingly, when each of the first body 10 and the second body 20 includes the curved part and bottom part, the curved part and bottom part may include a sliding coupling parts 130 or 230 configured as a pair of coupling protrusion 131 or 231 and fitting groove 132 or 232 which are slidably coupled to each other as illustrated in FIGS. 2 to 4. Additionally, the curved part and bottom part may include a fastening point combination part 140 or 240 which forms a fastening point at one point by coupling parts facing each other to each other which are located at other sides at which the pair of coupling protrusion 131 or 231 and fitting groove 132 or 232 is not formed. Referring to the exploded view of FIG. 4, at least one sliding coupling part 130 or 230 and at least one fastening point combination part 140 or 240 may be formed on each of the first body 10 and the second body 20. For example, the sliding coupling parts 130 and 230 may include respectively the coupling protrusions 131 and 231 protruding respectively toward the first bottom part 120 and the second bottom part 220 from the first curved part 110 and the second curved part 210, and the fitting grooves 132 and 232 having shapes of long holes formed respectively through the first bottom part 120 and the second bottom part 220 in the longitudinal directions described above. Accordingly, by slidingly inserting the coupling protrusion 131 or 231 into the fitting groove 132 or 232 and moving the coupling protrusion, each of the curved part and the bottom part may be coupled to each other very conveniently. After this coupling, the fastening point combination part 140 or 240 which forms the fastening point at one point may be used to more securely fasten each pair of curved parts and bottom parts to each other. For example, the fastening point combination part 140 or 240 may include hole parts 141 and 142 or 241 and 242 arranged in a pair at positions at which the curved part and the bottom part face each other. For example, a fastening member such as a screw (not shown) passes through the pair of hole parts (for example, 141 and 142) corresponding to each other to form a secure fastening point. As described above, due to the complex use of the structure of combining in a sliding manner and the fastening structure of forming the fastening point at one point, it is possible to conveniently and securely manufacture the first body 10 and the second body 20.

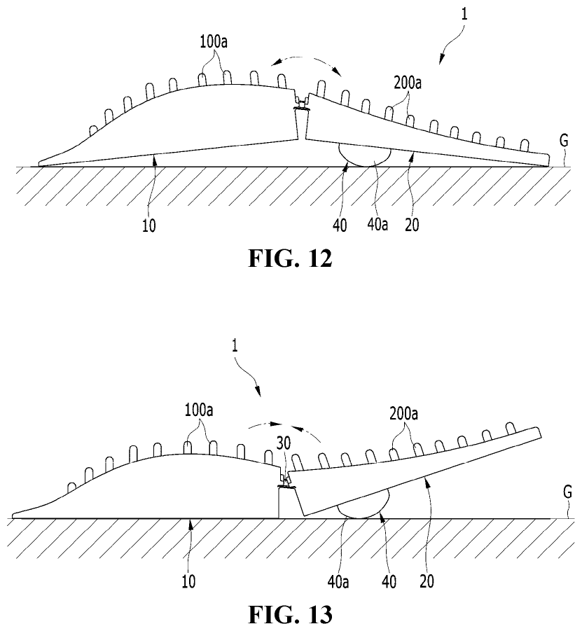

The removable rolling bar 40 is coupled removably to the first body 10 or the second body 20. As illustrated in FIGS. 2 to 4, the removable rolling bar 40 is coupled to the bottom surface of the first body 10 or the second body 20 having no protrusion and supports the first body 10 or the second body 20, and causes the first body 10 or the second body 20 to perform a seesaw movement relative to a support point thereof (see FIGS. 10 and 11). The seesaw movement induced by the removable rolling bar 40 and the bending of the flexible connector 30 located between the first body 10 and the second body 20 to be described later are combined to variously change the entire shape of the spinal orthopedic exercise equipment 1 such that the spinal orthopedic exercise equipment 1 can effectively stimulate the body. The flexible connector 30 will be described later in more detail.

The removable rolling bar 40 is formed in the form of a bar extending in one direction, and includes a curved outer circumferential surface 40a. The outer circumferential surface 40a of the removable rolling bar 40 may be configured as a convex curved surface such that the removable rolling bar 40 can easily roll. In the removable rolling bar 40, the outer circumferential surface 40a which is not in contact with the bottom surface [a flat surface constituted by the first bottom part 120 or the second bottom part 220 described above] of the first body 10 or the second body 20 may be configured as a convex curved surface. The removable rolling bar 40 can roll easily when the convex outer circumferential surface 40a thereof is supported by the ground. The curved surface of the outer circumferential surface 40a is not required to be particularly limited as long as the removable rolling bar 40 can roll easily.

The removable rolling bar 40 is removed as illustrated in FIG. 3 and may be easily coupled to any one of the first body 10 and the second body 20. The removable rolling bar 40 may be securely coupled to any one of the first body 10 and the second body 20 by combining a cylindrical coupling protrusion 410 protruding to a side with a cylindrical coupling hole 150 or 250 formed in the first body 10 or the second body 20. The removable rolling bar 40 may include the cylindrical coupling protrusion 410 protruding perpendicularly to the removable rolling bar 40 on a surface thereof in contact with the bottom surface of the first body 10 or the second body 20. As illustrated in FIG. 3, protruding perpendicularly to the removable rolling bar 40 means protruding perpendicularly to the longitudinal direction of the removable rolling bar 40. A surface in which the cylindrical coupling hole 150 or 250 is formed may be located at a side opposite to the outer circumferential surface 40a of the removable rolling bar 40 described above and may be formed in a flat shape rather than a curved surface. The cylindrical coupling hole 150 or 250 may be formed by protruding to have a cylindrical shape as illustrated in FIG. 3.

The cylindrical coupling holes 150 and 250 which receive the cylindrical coupling protrusion 410 may be formed respectively in the bottom surfaces of the first body 10 and the second body 20 [flat surfaces constituted respectively by the first bottom part 120 and the second bottom part 220]. The inner diameter of the cylindrical coupling hole 150 or 250 may not be smaller than the outer diameter of the cylindrical coupling hole 150 or 250. Accordingly, by using the cylindrical coupling protrusion 410, the removable rolling bar 40 may be easily coupled to the cylindrical coupling hole 150 or 250 formed in the first body 10 or the second body 20. After the coupling, the structure of stoppers 151 or 251 and 411 are used to arrange the removable rolling bar 40 perpendicularly to the longitudinal direction in which the first body 10 and the second body 20 are connected to each other such that the removable rolling bar 40 can be securely fastened. That is, the stoppers 151 or 251 and 411 may be formed respectively on the cylindrical coupling hole 150 or 250 and the outer side of the cylindrical coupling protrusion 410, the stoppers being engaged with each other to restrict the rotation of the removable rolling bar 40 and to fasten the removable rolling bar 40 perpendicularly to the longitudinal direction in which the first body 10 and the second body 20 are connected to each other. Specifically, referring to FIGS. 3 and 4, the stoppers 151 or 251 and 411 may include the stopper 411 of the cylindrical coupling protrusion 410 and the stopper 151 or 251 of the cylindrical coupling hole 150 or 250, the stopper 411 and the stopper 151 or 251 being configured in a pair. The stopper 411 of the cylindrical coupling protrusion 410 may be configured as a part protruding radially on the outer side of the cylindrical coupling protrusion 410, and the stopper 151 or 251 of the cylindrical coupling hole 150 or 250 may be formed as a part of the first bottom part 120 or the second bottom part 220 on the circumference of the cylindrical coupling hole 150 or 250 so as to have at least a portion of the stopper 151 or 251 machined in an angled shape such that the stopper 151 or 251 is in contact with the stopper 411. Additionally, an extension piece 412 may be formed on the stopper 411 of the cylindrical coupling protrusion 410 by protruding further to the outside, and an outside inlet hole 152 or 252 into which the extension piece 412 is inserted may be formed at a side of the circumference of the cylindrical coupling hole 150 or 250 at which the stopper 151 or 251 is not located.

Accordingly, the outside inlet hole 152 or 252 and the extension piece 412 are arranged to face each other, and the cylindrical coupling protrusion 410 of the removable rolling bar 40 is inserted into the cylindrical coupling hole 150 or 250 of the first body 10 or the second body 20, and when the removable rolling bar 40 is rotated relative to the cylindrical coupling protrusion 410, the extension piece 412 inserted into the outside inlet hole 152 or 252 is rotated to be held and fastened to the bottom surface of the first body 10 or the second body 20. In this case, when the stopper 411 of the cylindrical coupling protrusion 410 is in contact with the stopper 151 or 251 of the cylindrical coupling hole 150 or 250, the rotation of the removable rolling bar 40 stops, and the removable rolling bar 40, for example, may be fastened in a direction perpendicular to the longitudinal direction in which the first body 10 and the second body 20 are connected to each other as illustrated in FIG. 2. Accordingly, by using the stopper 411 and 151 or 152, the removable rolling bar 40 may be arranged in a desired direction on the first body 10 or the second body 20 to be coupled thereto.

Meanwhile, as illustrated in FIG. 3, an insertion protrusion part 413 arranged by being spaced radially apart from the cylindrical coupling protrusion 410 may be formed on a surface of the removable rolling bar 40 in contact with the bottom surface of the first body 10 or the second body 20. Additionally, an outer groove 153 or 253 may be formed on the bottom surface of the first body 10 or the second body 20 located at the periphery of the cylindrical coupling hole 150 or 250, the outer groove having a curved shape being located at a position corresponding to the insertion protrusion part 413 to receive the insertion protrusion part 413. The outer groove 153 or 253 receives the insertion protrusion part 413 such that the protrusion part 413 moves only therein, thereby restricting the rotation of the removable rolling bar 40 in which the insertion protrusion part 413 and fastening the removable rolling bar 40 in the corresponding position. Accordingly, due to the complex application of the structure of the stopper 411, 151, 152 described above and the structure in which the outer groove 153 or 253 and the insertion protrusion part 413 are formed, it is possible to more securely arrange and fasten the removable rolling bar 40 in a desired position. The removable rolling bar 40 is rotated in a direction contrary to a direction in which the removable rolling bar 40 is coupled to the first body 10 or the second body 20 relative to the cylindrical coupling protrusion 410 and thus can be easily removed from the first body 10 or the second body 20.

As illustrated in FIGS. 1 to 6, the flexible connector 30 is located between the first body 10 and the second body 20 to be connected thereto. The flexible connector 30 is provided between the first body 10 and the second body 20 to be bendably connected thereto such that the position of each of the first body 10 and the second body 20 can be dynamically changed (see FIG. 6). That is, the position of each of the first body 10 and the second body 20 described above may be changed according to the bending of the flexible connector 30, and due to the seesaw movement of the first body 10 or the second body 20 caused by the removable rolling bar 40, the arrangement of each of the first body 10 and the second body 20 can be more naturally changed (see FIGS. 10 and 11). The flexible connector 30 connects the first body 10 and the second body 20 to each other and may be embodied in various forms in which at least a part of the flexible connector 30 can be bent. The flexible connector 30 is not required to be limited to a specific structure or material, and may be variously modified within the limit that flexible connector 30 is located between the first body 10 and the second body 20 to be bendably connected thereto. However, in the present embodiment, the flexible connector 30 is configured as an elastic body which connects the curved surface of the first body 10 to the curved surface of the second body 20 by a continuous surface thereto, and the flexible connector 30, the first curved part 110 constituting the curved surface of the first body 10, and the second curved part 210 constituting the curved surface of the second body 20 may be configured to be integrated with each other. Hereinafter, the flexible connector 30 will be described in more detail on the basis of such an embodiment.

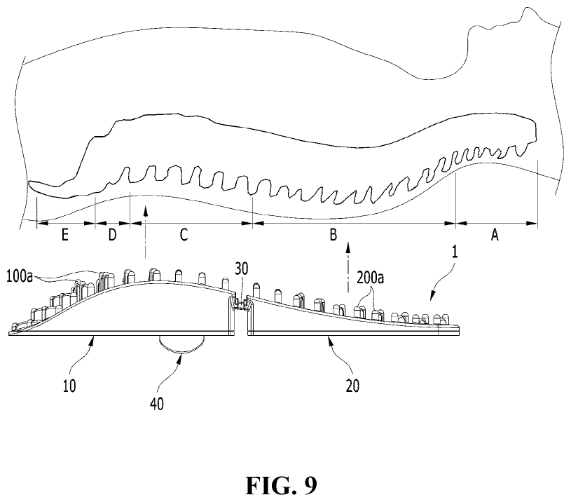

FIG. 8 is enlarged perspective views of the flexible connector of the exercise equipment in FIG. 1 when viewed in different directions. FIG. 8(a) is a view of the flexible connector viewed from a side at which protrusions of each body are formed, and FIG. 8(b) is a view of the flexible connector viewed from the bottom surface of each body.

As illustrated in FIGS. 8(a) and 8(b), the flexible connector 30 may be configured to include a curved surface located between the first body 10 and the second body 20 to be connected thereto. The flexible connector 30 may be configured as an elastic body which is elastically restored, and may bendably connect the first body 10 to the second body 20 due to such an elastic material of the flexible connector. Particularly, in the present embodiment, the flexible connector 30 may be configured to connect the first curved part 110 of the first body 10 to the second curved part 210 of the second body 20 by a continuous surface thereto, and due to such a structure, a smooth and three-dimensional curved surface may be formed from the first curved part 110 through the flexible connector 30 to the second curved part 210. The flexible connector 30 may be configured to be integrated with the first curved part 110 and the second curved part 210.

However, the flexible connector 30 is not necessarily limited in such a manner as described above and may be separately configured to be located between the first body 10 and the second body 20 to be coupled thereto.

As illustrated in FIGS. 8(a) and 8(b), the flexible connector 30 may include an outer indentation part 310 formed on each of the opposite end portions of the flexible connector which are not connected to the first body 10 and the second body 20 by being recessed inward from the outer side of the flexible connector. The outer indentation part 310 may reduce the amount of a curved surface passing through the flexible connector 30 between the first body 10 and the second body 20 such that the movement of the flexible connector 30 can be more efficiently performed. That is, due to the shape of a complex curved surface such as a horse saddle surface described above, the first body 10 and the second body 20 may not be easily bent in the longitudinal direction in which the first body 10 and the second body 20 are connected to each other, so the first body 10 and the second body 20 may be more efficiently bent in such a manner that a portion of the curved surface located between the first body 10 and the second body 20 to be connected thereto is removed in the width direction. Additionally, accordingly, the flexible connector 30 has the outer indentation part 310 to reduce the area of the flexible connector such that an elastic force exerted by the flexible connector 30 can be properly controlled. As described above, the first body 10 and the second body 20 between which there is no flexible connector 30 are spaced apart from each other, and accordingly, by using the associated space, the movement of the first body 10 and the second body 20 can be induced more efficiently.

FIG. 9 is a view illustrating the method of using the exercise equipment of FIG. 1, and FIGS. 10 to 13 are views illustrating the operation process of the exercise equipment of FIG. 1.

Hereinafter, the use examples and operation method of the spinal orthopedic exercise equipment having the above-described structural and functional characteristics will be described in more detail with reference to FIGS. 9 to 13.

The spinal orthopedic exercise equipment 1 may support a body in a state as illustrated in FIG. 9. That is, the spinal orthopedic exercise equipment 1 may support a user's back and waist and may apply pressure thereto by using protrusions [the first protrusions 100a and the second protrusions 200a]. In this case, a user's spine may be normally divided into parts at which the cervical vertebra A, the thoracic vertebra B, the lumbar vertebra C, the sacrum D, and the coccyx E are located. Particularly, the spinal orthopedic exercise equipment 1 may support at least a portion of the thoracic vertebra B, at least a portion of the lumbar vertebra C, and even parts corresponding to the sacrum D and the coccyx E connected to the lumbar vertebra C to apply pressure thereto. As illustrated in FIG. 9, the spinal orthopedic exercise equipment 1 of the present disclosure is placed under a user's waist and back while the user is lying down, so stimulation corresponding to the user's weight can be naturally applied to the user. The curved surface of the first body 10 and the curved surface of the second body 20 described above are connected to each other to have bending directions reversed to each other in the longitudinal direction (the longitudinal direction of the spine in the drawing) in which the first body 10 and the second body 20 are connected to each other, so a user's waist and the user's back connected to the waist can be in effective close contact with the curved surfaces having the reversed bending directions, respectively. That is, the curved surface of the first body 10 faces and supports the user's waist, and the curved surface of the second body 20 faces and supports the user's back, so the curved surface of the first body 10 and the curved surface of the second body 20 can effectively apply stimulation to the associated parts, respectively, by being in close contact therewith.

However, since this example is also not limiting, the method of using the spinal orthopedic exercise equipment 1 is not required to be limited thereto. That is, since this use example is also exemplary, the spinal orthopedic exercise equipment 1 may be disposed in a different form from the illustrated form to apply appropriate stimulation to a user. In this embodiment, as illustrated in FIG. 9, in a state in which the spinal orthopedic exercise equipment 1 is placed on the ground while a user is lying and the removable rolling bar 40 is in contact with the ground, the method of using the spinal orthopedic exercise equipment 1 will be described.

First, referring to FIGS. 10 and 11, in the spinal orthopedic exercise equipment 1, the removable rolling bar 40 may be mounted to the first body 10 to be used. The removable rolling bar 40 can easily roll by being in contact with the ground G due to the convex outer circumferential surface 40a. Accordingly, the removable rolling bar 40 supports the first body 10 relative to a position at which the removable rolling bar 40 is coupled to the first body 10, and can cause the first body 10 to perform a seesaw movement relative to the support point of the removable rolling bar 40. Accordingly, for example, the flexible connector 30 connected to the first body 10 unfolds as illustrated in FIG. 10, or folds in a direction contrary to the direction of the unfolding as illustrated in FIG. 11 such that the first body 10 and the second body 20 bend in directions contrary to each other, and thus the positions of the first body 10 and the second body 20 may change by corresponding thereto. That is, the positions of the first body 10 and the second body 20 may be changed according to the bending of the flexible connector 30. Such movements may be easily performed in such a manner that a user moves his or her waist or pushes the ground G with the user's feet or arms. Accordingly, the spinal orthopedic exercise equipment 1 very dynamically applies pressure to a user's waist and back to stimulate the user's spine. Accordingly, for example, it is possible to correct a part such as a posterior zygapophysis and eliminate or alleviate pain thereof.

In addition, as illustrated in FIGS. 12 and 13, the spinal orthopedic exercise equipment 1 may be used by mounting the removable rolling bar 40 to the second body 20. Even in such a case, by being in contact with the ground G, the removable rolling bar 40 can easily roll due to the convex outer circumferential surface 40a. Accordingly, the removable rolling bar 40 supports the second body 20 relative to a position to which the removable rolling bar 40 is coupled, and can cause the second body 20 to perform a seesaw movement relative to a support point thereof. Accordingly, the flexible connector 30 connected to the second body 20 unfolds as illustrated in FIG. 12, or folds in a direction contrary to the direction of the unfolding as illustrated in FIG. 13 such that the first body 10 and the second body 20 bend in directions contrary to each other, and thus the positions of the first body 10 and the second body 20 may change by corresponding thereto. Such movements may be easily performed in such a manner that a user moves his or her waist or pushes the ground G with the user's feet or arms. Accordingly, the spinal orthopedic exercise equipment 1 very dynamically applies pressure to a user's waist and back to stimulate the user's spine. In the case of FIGS. 10 and 11, and in the case of FIGS. 12 and 13, a part which performs a relatively large movement changes to the first body 10 or the second body 20. In such a manner, the different movements of the first body 10 and the second body 20 and the change of the positions thereof are induced such that new stimulation can be applied to different parts of a user's spine. Additionally, in each of the cases, due to the seesaw movement of the removable rolling bar 40, the flexible connector 30 folds or unfolds, and the moving directions of the first protrusions 100a and the second protrusions 200a of the bodies change greatly, so various types of stimulation can be applied to a wider range. In this manner, various types of exercises related to spinal orthopedic can be easily performed by using the spinal orthopedic exercise equipment 1. Furthermore, as described above, the spinal orthopedic exercise equipment 1 of the present disclosure has a simple structure and a small size, and thus is easy to be carried, so the spinal orthopedic exercise equipment 1 can be used by being moved from place to place and be conveniently used even at home without a separate installation process.

Although the embodiment of the present disclosure has been described with reference to the accompanying drawings, those skilled in the art to which the present disclosure pertains will understand that the spinal orthopedic exercise equipment of the present disclosure may be embodied in other specific forms without changing the technical spirit or essential characteristics thereof. Therefore, it should be understood that the embodiment described above is illustrative in all respects and not restrictive.

### DESCRIPTION OF THE REFERENCE NUMERALS IN THE DRAWINGS

1: Spinal orthopedic exercise equipment 10: First body
11, 21: Hollow part 20: Second body
30: Flexible connector 40: Removable rolling bar
40a: Outer circumferential surface 100a: First protrusion
100b, 200b: Connection plate 110: First curved part
120: First bottom part 130, 230: Sliding coupling part
131, 231: Coupling protrusion 132, 232: Fitting groove
140, 240: Fastening point combination part 141, 142, 241, 242: Hole part
150, 250: Cylindrical coupling hole 151, 251, 411: Stopper
152, 252: Outside inlet hole 153, 253: Outer groove
200a: Second protrusion 210: Second curved part
220: Second bottom part 310: Outer indentation part
410: Cylindrical coupling protrusion 412: Extension piece
413: Insertion protrusion part A: Cervical vertebra
B: Thoracic vertebra C: Lumbar vertebra
D: Sacrum E: Coccyx
G: Ground

### INDUSTRIAL APPLICABILITY

The present disclosure has the advantage of providing the spinal orthopedic exercise equipment which can obtain an improved exercise effect related to spinal orthopedics with a simple structure, and has industrial applicability.

What is claimed is:

1. Spinal orthopedic exercise equipment supporting a user's back and waist to apply pressure thereto, the equipment comprising:
a first body having a plurality of first protrusions protruding therefrom, a surface of the first body on which the first protrusions are disposed being configured as a curved surface,
a second body coupled to the first body and having a plurality of second protrusions protruding therefrom, a surface of the second body on which the second protrusions are disposed being configured as a curved surface different from the curved surface of the first body,
a flexible connector located between the first body and the second body to be bendably coupled thereto, and
a removable rolling bar coupled to a bottom surface of the first body or the second body having no protrusion and supporting the first body or the second body, the removable rolling bar causing the first body or the second body to perform a seesaw movement relative to a support point thereof.

2. The equipment of claim 1, wherein a position of each of the first body and the second body changes according to the bending of the flexible connector.

3. The equipment of claim 1, wherein the first body comprises a convex surface, and the second body comprises a concave surface, wherein bending directions of the curved surfaces of the first body and the second body are reversed to each other along a longitudinal direction in which the first body and the second body are connected to each other.

4. The equipment of claim 3, wherein the curved surface of the first body faces and supports a user's waist, and the curved surface of the second body faces and supports the user's back.

5. The equipment of claim 1, wherein the flexible connector is configured as an elastic body which connects the curved surface of the first body to the curved surface of the second body by a continuous surface thereto.

6. The equipment of claim 5, wherein the flexible connector, a first curved part in which the curved surface of the first body is formed, and a second curved part in which the curved surface of the second body is formed are configured to be integrated with each other.

7. The equipment of claim 1, wherein each of the first body and the second body comprises a curved part having a curved surface formed thereon and having protrusions protruding therefrom, and a bottom part coupled to the curved part and constituting a bottom surface having no protrusion,
wherein the curved part and the bottom part comprise a sliding coupling part configured as a pair of coupling protrusion and fitting groove which are slidably coupled to each other.

8. The equipment of claim 7, wherein the curved part and the bottom part further comprise a fastening point combination part which forms a fastening point at one point by coupling parts facing each other to each other which are located at other sides at which the pair of coupling protrusion and fitting groove is not formed.

9. The equipment of claim 1, wherein the removable rolling bar has an outer circumferential surface configured as a convex curved surface, wherein the outer circumferential surface is not in contact with the bottom surface of the first body or the second body.

10. The equipment of claim 9, wherein the removable rolling bar comprises a cylindrical coupling protrusion protruding perpendicularly to the removable rolling bar on a surface thereof in contact with the bottom surface of the first body or the second body.

11. The equipment of claim 10, further comprising:
a cylindrical coupling hole formed in the bottom surface of each of the first body and the second body and receiving the cylindrical coupling protrusion, and
stoppers formed respectively on the cylindrical coupling hole and an outer side of the cylindrical coupling protrusion, the stoppers being engaged with each other to restrict a rotation of the removable rolling bar and to fasten the removable rolling bar perpendicularly to a longitudinal direction in which the first body and the second body are connected to each other.

12. The equipment of claim 1, wherein the first body and the second body comprise respectively hollow parts which are spaces in which protrusions are not formed, the hollow parts being located respectively between the first protrusions and between the second protrusions and respectively along centers of the first body and the second body in a longitudinal direction in which the first body and the second body are connected to each other.

13. The equipment of claim 1, further comprising:
a plurality of connection plates arranged by intersecting with a longitudinal direction in which the first body and the second body are connected to each other, each of the connection plates formed between the first protrusions and between the second protrusions so as to connect at least two protrusions of the first protrusions to each other and to connect at least two protrusions of the second protrusions to each other.
